(19) United States
(12) Patent Application Publication
(10) Pub. No.: US 2023/0233644 A1
(43) Pub. Date: Jul. 27, 2023

(54) PROCESS FOR THE REMOVAL AND RECOVERY OF HESPERALOE EXTRACTIVES

(71) Applicant: KIMBERLY-CLARK WORLDWIDE, INC., Neenah, WI (US)

(72) Inventors: Ning Wei, Roswell, GA (US); Thomas G. Shannon, Neenah, WI (US); Brent M. Thompson, Oshkosh, WI (US); Gregory J. Wideman, Menasha, WI (US)

(21) Appl. No.: 18/011,310

(22) PCT Filed: Jun. 21, 2021
(86) PCT No.: PCT/US21/38270

§ 371 Date: Dec. 19, 2022

Related U.S. Application Data
(60) Provisional application No. 63/041,220, filed on Jun. 19, 2020.

(51) Int. Cl. A61K 36/88; B01D 11/02; B01D 9/00; A61K 31/7048

(52) U.S. Cl. CPC A61K 36/88; B01D 11/0288; B01D 9/0054; B01D 11/0284; A61K 31/7048; A61K 2236/37; A61K 2236/331; A61K 2236/333; A61K 2236/35

(57) ABSTRACT

Disclosed are soluble extractives prepared from non-woody plants of the genus Hesperaloe and processes for preparing the same. The extracts preferably comprise at least one saponin. In certain instances, the process includes providing biomass derived from non-woody plants of the genus Hesperaloe, milling the biomass, washing the biomass with a solvent to yield a crude extract and optionally further purifying the crude extract by filtration to remove water insoluble compositions such as fibers, fines, epidermal debris and lipids. Preferably, the composition extracted from Hesperaloe comprises 25(27)-dehydrofucreastatin, 5(6),25(27)-disdehydroyuccaloiside C, 5(6)-disdehydroyuccaloiside, C, furcreastatin, yuccaloiside, or a mixture thereof.

### BACKGROUND

Plants produce a vast and diverse assortment of organic compounds, the great majority of which do not appear to participate directly in their growth and development. These substances, traditionally referred to as secondary metabolites or plant natural products, often are distributed among limited taxonomic groups within the plant kingdom. The functions of secondary metabolites remain largely unknown, although a number of compounds have been associated with attributes useful to the plants e.g. protection against herbivores and protection against microbial infection, as attractants for pollinators and seed-dispersing animals, and as compounds that influence competition among plant species (allelochemicals). There is a growing interest in plant natural products, since these products often have a wide range of applications in different kinds of industries, including pharmaceutical industries, cosmetic industries, food industries, detergent industries, and the like.

A particular group of plant secondary metabolites of interest are saponins. Saponins are glycosylated compounds classified as either triterpenoids, steroids, or steroidal glycoalkaloids. Saponins consist of one or two sugar moieties which are coupled to the aglycon (mono- and bisdesmosides, respectively). Saponins can be hydrolyzed to sapogenins and sugar moieties by acid hydrolysis or enzymatic methods. Saponins are generally water soluble high molecular weight compounds with molecular weights ranging from 600 to more than 2,000 daltons.

The asymmetric distribution of their hydrophobic (aglycone) and hydrophilic (sugar) moieties confers an amphipathic character to these compounds which are largely responsible for their detergent-like properties. The ability of lowering surface tension make saponins potentially well suited for use in the cosmetic and in the detergent industries.

Saponins also have the ability of forming insoluble complexes with cholesterol, which makes some of them suitable for use in the pharmaceutical industry as cholesterol lowering agents. Other saponins are associated with formation of immunostimulating complexes that are useful in vaccine strategies.

Currently, a major limitation to the broad exploitation of saponins is the fact that commercially available saponins are relatively expensive. The expenses is due in large part to the limited number of plant extracts having significant amounts of saponins. Currently, commercially available plant extracts containing saponins include Saponaria officinalis, Quillaia bark and stem, Castanea sativa seeds, and extracts of various Yucca species.

Plant extracts containing saponins are thus of general interest within a wide range of different industries. There is therefore a growing need in the art for alternative sources of saponin extracts and these plant sources should preferably be cheap, easy to obtain, and preferably the saponin content should be relatively high.

### SUMMARY

The present inventors have now discovered that Hesperaloe biomass, particularly the above ground portion of a Hesperaloe plant and more particularly the portion of the Hesperaloe plant above the crown, may be processed to extract solids, particularly water soluble solids, such as inorganic salts, saccharides and saponins. In certain instances, the extraction may comprise milling biomass derived from a non-woody plants of the genus Hesperaloe to yield a crude extract, also referred to herein as a juice, which may be further processed to concentrate or isolate specific water soluble solids. In other instances, the extraction process may comprise milling the biomass and washing the milled biomass with an aqueous solvent to yield a juice, separating water insoluble solids from the juice, and optionally concentrating the juice.

Accordingly, in one embodiment the present invention provides a method of processing biomass derived from non-woody plants of the genus Hesperaloe including, for example, Hesperaloe funifera, Hesperaloe nocturna, Hesperaloe parviflora, and Hesperaloe chiangii to minimize the damage to the cellulosic fibers, such as by minimizing the shortening of the fibers by cutting or excessive fibrillation and formation of fines by grinding, while extracting hydrophilic compounds from the biomass. In certain instances, the processing may comprise milling the biomass by mechanically applying pressure to the biomass using a tandem mill or a screw press. In other instances, it may comprise non-mechanical means such as diffusion. For example, in one particularly preferred embodiment, the invention provides a process for preparing an extract from Hesperaloe biomass comprising the steps of cutting the biomass, milling the biomass, extracting the biomass with a solvent, imbibing the biomass with juice, and depithing the biomass.

In other embodiments, the present invention provides a method of processing biomass derived from a non-woody plants of the genus Hesperaloe through a series of mills, such as two, three, four, five, six or seven mills, optionally with imbibition and/or depithing, to remove a water soluble fraction of the biomass. The water soluble fraction may comprise saccharides, polysaccharides, inorganic salts, saponins and sapogenins. In certain instances, the water soluble fraction may be recovered from the milling process as a juice that may be subjected to further processing to yield a composition comprising one or more of inorganic salts, saccharides, saponins or sapogenins in purified or substantially pure form.

In other embodiments, the present invention provides a method of extracting biomass derived from the leaves of non-woody plants of the genus Hesperaloe prior to pulping comprising the steps of milling the biomass, extracting the biomass with an aqueous solvent, and recovering water soluble solids from the aqueous solvent. In a particularly preferred embodiment, the extraction process does not rely upon the addition of alkali such as sodium hydroxide (NaOH), sodium carbonate (Na₂CO₃), sodium bicarbonate (NaHCO₃) potassium hydroxide (KOH), potassium carbonate (K₂CO₃), potassium bicarbonate (KHCO₃), ammonium hydroxide (NH₄OH) or combinations thereof. Rather, the extraction process may be carried out with an aqueous solvent, such as water, having a pH from about 5 to about 9. In certain instances, imbibition water (e.g., dilute juice) can be added in one or more of the milling steps to increase extraction of water soluble solids from the biomass. In this manner, the present invention seeks to remove high value water soluble solids prior to pulping, which commonly employs caustic chemicals that significantly degrade the water soluble solids, so as to maximize the value of the entire biomass.

In still other embodiments the present invention provides a Hesperaloe extract comprising at least one, preferably two or more, and still more preferably three or more, water soluble solids selected from inorganic salts, saccharides and saponins. In a particularly preferred embodiment, the extract is subjected to further purification, such as filtration, to remove water insoluble solids such that the extract composition is substantially free from water insoluble solids. In certain instances, the extraction processes of the present invention remove at least about at least about 50% of the water soluble solids from the biomass, such as from about 50 to about 98%, such as from about 60 to about 90%. In this manner, the amount of water soluble solids removed from the biomass, based upon the bone dry weight of the biomass, may be at least about 10 wt %, more preferably at least about 20 wt % and still more preferably at least about 25 wt %, such as from about 10 to about 40 wt %, such as from about 15 to about 35 wt %, such as from about 20 to about 30 wt %.

In other embodiments, the present invention provides an aqueous composition extracted from non-woody plants of the genus Hesperaloe comprising from about 2 to about 10 wt % saccharides, from about 15 to about 25 wt % saponin, from about 0 to about 5 wt % protein, from about 0 to about 5 wt % lipids and from about 5 to about 25 wt % inorganic salts, where the weight percentages are based upon the total weight water soluble solids.

In certain instances, the composition may be concentrated and treated to remove impurities to yield a composition comprising saponins as its chief component. For example, in certain embodiments, the invention provides a process for preparing a substantially pure saponin composition comprising the steps of mixing the juice with a salt and a solvent to form a first solution, adjusting the pH of the first solution to about 6.0 to about 7.0, adding at least one phosphate to the first solution to form an ion-polysaccharides precipitated and removing the precipitated, such as by filtration, to yield a substantially pure saponin composition. In certain instances, the first solution may be heated to facilitate precipitation of the ion-polysaccharides complexes

In still other embodiments, the present invention provides a substantially pure saponin composition is substantially free from phenolic compounds (such as tannins, quercetin, leucocyanidin, kaempferol, among others), organic acids (such as caffeic acid, gallic acid, coumaric acid), free saccharides, lipids, and nitrogen-containing compounds, such as proteins. The terms free polysaccharide and free phenolic compounds generally refer to compounds that are not part of a saponin. Thus, while saponins may contain one or more saccharides, such saccharides are not “free” saccharides.

### DESCRIPTION OF THE DRAWINGS

FIGS. 1A and 1B illustrate a triterpenoid saponin and a steroidal saponin, respectively;

FIG. 2 illustrates one embodiment of a milling process useful in the present invention;

FIG. 3 illustrates another embodiment of a milling process useful in the present invention;

FIGS. 4A-C illustrate various novel saponins extracted from non-woody plants of the genus Hesperaloe according to the present invention including, 25(27)-dehydrofucreastatin (FIG. 4A), 5(6),25(27)-disdehydroyuccaloiside C (FIG. 4B), and 5(6)-disdehydroyuccaloiside C (FIG. 4C);

FIG. 5 is a schematic of the extraction of Hesperaloe funifera as described in the Example;

FIG. 6 is a schematic of the extraction of CHCl3-MeOH fraction as described in the Example;

FIG. 7 is a schematic of the extraction of MeOH fraction as described in the Example;

FIG. 8 is a schematic of the extraction of MeOH-water fraction as described in the Example; and

FIG. 9 is a schematic of the extraction of hot water fraction as described in the Example.

### DEFINITIONS

As used herein the term “biomass” generally refers to whole plants and plant organs (i.e., leaves, stems, flowers, roots, etc.) of the genus Hesperaloe including, for example, Hesperaloe funifera, Hesperaloe nocturna, Hesperaloe parviflora, and Hesperaloe chiangii. In particularly preferred instances, water soluble solids may be prepared from biomass consisting essentially of the above ground portion of the Hesperaloe plant and more particularly the portion of the Hesperaloe plant above the crown and still more preferable the leaves of the Hesperaloe plant.

As used herein the term “bagasse” generally refers to biomass that has been subjected to an extraction process such as, for example, continuous solvent extraction or milling, so that the resulting solids have less water soluble solids than the biomass from which it is derived. In certain preferred embodiments bagasse is prepared by subjecting biomass to high pressure, such as by milling. High pressure may be achieved by using compression pressure, such as that provided by machines such one or more opposed counter-rotating rolls, a mechanical press, a screw press as well as by direct hydraulic pressure and other processes to apply pressure to the biomass and remove intercellular and intracellular liquid.

As used herein the term “fines” generally refers to fibrous water insoluble cellulosic material having a length to width aspect ratio of from about 1 to about 100 and wherein the length of the fibrous water insoluble material is less than about 0.2 mm.

As used herein the term “milling” generally refers to the application of sufficient pressure to force the intercellular and intracellular liquid from the biomass.

As used herein, the term “saccharide” is used interchangeably with the terms “polysaccharide,” “oligosaccharide” and “sugar” the definitions of which are well known to those skilled in the art of carbohydrate chemistry. It should be noted that the saccharides can be in the form of mono-, oligo- and/or polysaccharides. Preferably saccharides are water soluble and do not include cellulose, hemicellulose or mono-, oligo- and/or polysaccharides bound to other compounds, such as glycosides (arabinose, glucose, galactose, xylose, and glucuronic acid) bound to a triterpenoid to form a saponin.

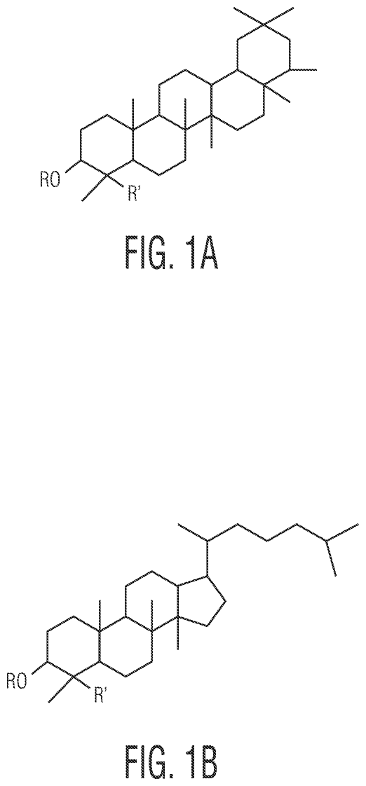

As used herein the term “saponin” generally refers to glycosides comprising a sugar component referred to as a glycone and a non-sugar component referred to as an aglycone. Depending on the structure of the aglycone the saponin may be classified as a triterpenoid saponin, illustrated in FIG. 1A, or to steroidal saponin, illustrated in FIG. 1B. The aglycone portion of the saponin may be either a pentacyclic triterpenoid or a tetracyclic triterpenoid, both of which contain 30 carbon atoms. Whether steroidal or triterpenoid, saponins may be mono, bi- or tridesmodic. Monodesmodic saponins have a single saccharide, normally attached at C-3. Bidesmodic saponins have two saccharides, often with one attached through an ether linkage at C-3 and the other either attached through an ester linkage at C-28 or through an ether linkage at C-20 (pentacyclic and tetracyclic triterpene saponins, respectively), or through an ether linkage at C-26 (furostane saponins). In certain instances, Hesperaloe biomass may comprises at least about 5 wt % of total saponins, such as from about 5 to about 15 wt %, such as from about 8 to about 12 wt %, based upon the bone dry weight of the biomass. Total saponins may be determined as described in the Test Methods section below.

As used herein the term “water soluble solids” generally refers to dry matter which remains after the extract has been centrifuged, filtered and all water is evaporated. The procedure for measuring water soluble solids of a biomass extract of the present invention is described in detail in the Test Methods section below. Water soluble solids may be expressed on a percentage basis relative to the mass of bone dry biomass.

As used herein the term “water insoluble solids” generally refer to the fraction of extract that is removed by centrifugation and filtration in the course of measuring water soluble solids, as described in the Test Methods section below.

### DETAILED DESCRIPTION

This invention relates to extractives and processes to enhance the recovery of the same from non-woody plants and more particularly the non-woody plants of the genus Hesperaloe. In particular, the present invention is directed towards processing biomass derived from non-woody plants of the genus Hesperaloe including, for example, Hesperaloe funifera, Hesperaloe nocturna, Hesperaloe parviflora, and Hesperaloe chiangii to remove water soluble solids. Water soluble solids that may be recovered according to the present invention include, for example, saccharides, polysaccharides, inorganic salts, saponins and sapogenins.

Extractives may be recovered from non-woody plants of the genus Hesperaloe by extracting biomass, particularly the leaves and more particularly the leaves above the crown of the plant, with at least one solvent selected from the group consisting of water, methanol, ethanol, butanol, and isopropanol and mixtures thereof. For example, in one embodiment, the process comprises contacting biomass with an extractant solution comprising water and separating the water soluble fraction from the insoluble biomass fraction. In other embodiments the extractant solution may comprise, in addition to water, a surfactant, a solvent and optionally extract-bearing juice. The extract-bearing juice can come from, for example, an earlier extraction step or an earlier milling step.

A simple water extraction of Hesperaloe biomass may yield a crude aqueous extract comprising saccharides, polysaccharides, inorganic salts, saponins and sapogenins. A crude extract may also be produced using methanol as a solvent, or a mixture of methanol and water, to extract biomass, which may have been previously extracted with acetone or diethyl ether to remove lipids and pigments. In other instances, the biomass may be extracted with a 4:1 ethanol-water solvent, followed by subsequent defatting of the extract with a non-polar solvent such as hexane. In certain instances, the defatted extract may be subjected to further treatment to isolate specific water soluble components, such as saponins, which may be purified from the defatted extract by mixing with butanol and separating the butanol phase to yield a mixture of saponins that are substantially free from proteins and free saccharides and polysaccharides.

Hot aqueous extractants can also be used. For example, in one embodiment water soluble solids may be extracted from Hesperaloe biomass, particularly the leaves, by extracting the biomass with hot aqueous ethanol or isopropanol (75 to 95% by weight alcohol). The aqueous alcohol extraction fluid may then be filtered and concentrated, and the fat-soluble material may be removed by mixing the extraction fluid with a non-polar solvent such as hexane. A substantially pure saponin composition may then be prepared by further extracting defatted extract with a polar solvent such as butanol.

In still other embodiments saponins may be extracted from Hesperaloe biomass by milling the biomass at extracting at room temperature with a solution of 90% acetonitrile in water with sonication followed by filtration and removal of the solvent under vacuum. Saponins may be further purified by high performance liquid chromatography using CHCl₃-MeOH-water (4:4:2 v/v) to yield various fractions that may be recrystallized from MeOH. In certain instances, saponins maybe purified using the serial extraction and fraction techniques described in the Examples below.

For the purpose of preparing the compositions of the present invention, and for use in the present method, a simple aqueous extract may be preferred, although other extraction methods are within the scope of the present invention. In a particularly preferred embodiment, Hesperaloe biomass may be cut to size, pressed, and extracted with an aqueous solvent to remove water soluble extracts such as inorganic salts, saccharides, polysaccharides, organic acids and saponins. The water soluble extracts are collected and may be concentrated by techniques well known in the art such as, for example, evaporation, spray-drying, drum drying and the like. The extract may be concentrated until it has a solids content of about 20 to about 100% solids by weight, such as from about 20 to about 95% solids by weight, such as from about 20 to about 80% solids by weight.

In a particularly preferred embodiment water soluble extracts are concentrated by spray drying by feeding the extract solution to atomizing equipment. Suitable atomizing equipment includes, but is not limited to, a rotary wheel atomizer, a pressure nozzle atomizer, and a dual fluid nozzle atomizer. Rotary wheel, pressure nozzle and dual fluid nozzle atomizers are known to those of ordinary skill in the art and include those in spray dryers commercially available from a variety of sources, such as GEA Process Engineering.

As will be described in more detail below, the biomass may be milled to separate the bagasse and water soluble solids using a roll, screw, and other forms of presses. In certain preferred embodiments biomass is passed between one or more nips of opposed counter-rotating rolls to maximize the mechanical removal of juice. The bagasse can then be contacted with the juice in a subsequent milling step, as will be described more fully below. In certain instances, the biomass may be cut to size and cleaned prior to milling. Cutting and cleaning may be carried out using well known methods in the art. In a particularly preferred embodiment, the biomass is cleaned to remove debris such as dirt without the use of water or other solvents. While it may be preferable to cut the biomass to size prior to extraction, it is generally preferred not to grind, pulp, shred or macerate the biomass before it is milled. While such physical processing steps can be advantageous in that they expose more of the biomass surface to the extractant solution, they can break the plant cell walls, excessively shorten fiber length, and create an excessive amount of fines. It is generally desirable to avoid negatively affecting the bagasse in this manner during the extraction phase. In this manner, the extraction method of the present invention typically yields bagasse fiber that may be further processed, such as by pulping, to yield a pulp fiber well suited for the manufacture of paper products.

In other embodiments the water soluble solids may be recovered from biomass by diffusion. In diffusion, the biomass brought into contact with the liquid to extract the liquid components. Usually, the biomass is prepared by first cutting, but not shearing or crushing so as to minimize the damage to fibers and avoid the creation of an excessive amount of fines. The prepared biomass is then washed repeatedly, usually using a solvent, to extract the liquid contained in the biomass. The solvent can be any of the foregoing solvents. An exemplary treatment solvent is water, particularly hot water such as water heated to a temperature from about 40 to about 90° C. The solvent can be circulated and reused so that the solvent used for a first extraction is reused as a solvent to extract subsequent prepared biomass.

Various types of diffusers are known in the art and can be adapted for use with biomass as described herein. Suitable diffusers include a ring diffuser, a tower diffuser, or a drum diffuser. Exemplary diffusion systems are discussed, for example, in U.S. Pat. Nos. 4,182,632, 4,751,060, 5,885,539 and 6,193,805 the contents of which are hereby incorporated in a manner consistent with the present disclosure. Numerous other diffusion methods and devices for the diffusion method are known and can be adapted for use in the methods described herein. One such diffuser is the continuous-loop, counter-current, shallow-bed Crown Model III Percolation Extractor, commercially available from Crown Iron Works, Blaine, Minn.

In a specific embodiment the biomass may be cut to size prior to extraction. For example, the biomass may be cut to size at the time of harvesting using a forage harvester. A forage harvester typically comprises a header and a cutter wheel or drum. In a preferred embodiment the biomass is cut directly by the harvester header, using reciprocating knives, disc or rotary mowers or large saw-like blades. The header is configured such that the cut height is above the crown of the plant such as from about 4 inches to about 12 inches above the ground. From the header the biomass is fed to the cutter wheel. The cutter wheel is equipped with several knives fixed to it that chops and blows the silage out a chute of the harvester into a wagon that is either connected to the harvester or to another vehicle driving alongside. The configuration of the knives, the number of knives attached to the cutter wheel and the speed of the cutter wheel determines the cut size of the biomass. In one embodiment, the biomass size is selected such that the nominal chop length is from 5 to about 100 mm, such as from about 10 to about 80 mm, such as from about 20 to about 60 mm. It should be noted that the nominal chop length is set by the harvester and the actual chop length of the material may vary depending upon the consistency of orientation of the biomass feeding into the cutter wheel as well as other factors.

In another embodiment, the biomass may be reduced via a hammermill. For example, the harvested biomass may be modified into a format that can be handled more easily by the hammermill operation using such things as tub grinders, horizontal grinders/shredders, or simple woodchippers. These first stage systems typically have large rotating drums with large blunt hammers that quickly shear or shred the material into a less dense, loose format that can be easily milled to the desired size. Large screens are generally used in first-stage grinding to prevent oversized material from exiting the grinding chamber. These screens may range in size from about 5 to about 15 cm openings. Chippers typically use rotating drums with fixed knives parallel to the drum axis. Chip size is generally controlled by feed rate with the chip size ranging from about 0.5 to about 5 cm. Once the first-stage grinding or chipping is completed, the feedstock is milled to the desired particle size using a hammer mill. Hammer mills use large rotating drums with protruding metal bars (i.e., hammers) that impact the material at high velocity to shatter and tear material particles. Typically, the metal bars swing freely from the drum, but fixed hammers are also common in hammer mill designs. The size of biomass exiting the hammermill may range from about 1.0 to about 10 mm. The particle size may be varied within the desired range of 1.0 to about 10 mm depending upon the desired separation efficiency or end use of the bagasse.

The biomass, cut or uncut, may be extracted by any suitable extraction process as discussed above. In a particularly preferred embodiment, the solvent used for extraction comprises water. One of skill in the art will recognize the ratio of extraction solvent to biomass will vary based on the solvent, the amount of biomass to be extracted and the extraction procedure. In certain preferred embodiments, the extraction solvent is water and the ratio of extraction solvent to biomass, on the basis of liters of extraction solvent to kilogram of bone-dry biomass, is from about 1:5 to about 1:100, such as from about 1:5 to about 1:50 and more preferably from about 1:5 to about 1:20.

The pH of the extraction solvent can be between about pH 5.0 and 8.0, such as, for example, between about pH 6.0 and about pH 8.0, between about pH 6.5 and about pH 7.5. In a particular embodiment, the extraction solvent is water having a pH between about pH 6.5 and about pH 7.5. In those embodiments where extraction includes imbibition with a crude juice, the imbibition fluid may have a pH from about 4.0 to about 5.0.

The extraction may be carried out at temperatures between about 25 and about 90° C., such as, for example, between about 30 and about 80° C., between about 35 and about 75° C., between about 40 and about 70° C., between about 45 and about 65° C. or between about 50 and about 60° C.

In embodiments where the extraction process is a batch extraction process, the duration of extraction may range from about 0.25 to about 24 hours, such as, for example, from about 0.5 to about 2 hours, from about 1 to about 8 hours, or from about 1 to about 6 hours.

In embodiments where the extraction process is a continuous process, the duration of extraction may range from about 0.25 to about 5 hours, such as, for example, from about 0.5 to about 3 hours.

After extraction the water insoluble biomass material may be separated from the water soluble solids by filtration to provide a filtrate containing inorganic salts, saccharides, polysaccharides, organic acids and saponins (referred to herein as the “first filtrate”). Separation can be achieved by any suitable means including, but not limited to, gravity filtration, a plate-and-frame filter press, cross flow filters, screen filters, Nutsche filters, belt filters, ceramic filters, membrane filters, microfilters, nanofilters, ultrafilters or centrifugation. Optionally various filtration aids such as diatomaceous earth, bentonite, zeolite, etc., may also be used in this process.

After separation, the pH of the first filtrate may be adjusted to remove additional impurities. In one embodiment, the pH of the first filtrate can be adjusted to between about 8.5 and about 10.0 by treatment with a base, such as, for example, calcium oxide or hydroxide (about 1.0% from the volume of filtrate) with slow agitation.

In a particularly preferred embodiment, water soluble solids are removed from biomass, particularly Hesperaloe leaves, prior to pulping by a series of mills, such as two, three, four, five, six or seven mills arranged in tandem, optionally with imbibition and/or depithing. Generally, processing biomass according to the present invention removes at least about 25% of the water soluble solids from the biomass, more preferably at least about 50%, still more preferably at least about 75%, such as from about 25 to about 98%, such as from about 50 to about 90%, such as from about 75 to about 90%.

The amount of water soluble solids recovered from biomass may vary depending on the extraction efficiency, however, in certain instances from about 100 to about 400 grams of water soluble solids may be extracted per kilogram of bone dry biomass, such as from about 120 to about 350 grams per kilogram, such as from about 150 to about 300 grams per kilogram. Of the extracted water soluble solids, the total saponins may comprise at least about 5 wt %, such as at least 10 wt %, such as at least 20 wt %, such as from about 5 to about 40 wt %, such as from about 10 to about 30 wt %, based upon the bone dry weight of the water soluble solids. In certain instances the amount of total saponins that may be extracted from biomass may range from about 10 to about 400 grams per bone dry kilogram of biomass, such as from about 20 to about 300 grams, such as from about 25 to about 200 grams, such as from about 10 to about 100 grams. In certain instances, the amounts of materials (on bone dry grams per kilogram of bone dry biomass) removed from the biomass during the extraction process may range as set forth in Table 1, below.

TABLE 1

| | Amount |
|---|---|
| | (g/kg of bone dry biomass) |
| Total Extracted Solids | 100-400 |
| Total Water Insoluble Solids | 5-50 |
| Total Water Soluble Solids | 95-350 |
| Total Saponins | 5-160 |

In addition to saponins, the water soluble solids may comprise saccharides, proteins, lipids, and inorganic salts. For example, in certain instances, the water soluble solids may comprise from at least about 1 wt %, based upon the bone dry weight of water soluble solids, saccharides, such as from about 1 to about 15 wt %, such as from about 2 to about 10 wt %. The saccharides may comprise monosaccharides and oligosaccharides. In other instances, the water soluble solids may comprise from at least about 15 wt %, based upon the bone dry weight of water soluble solids, inorganic salts, such as from about 15 to about 30 wt %.

In other embodiments, aqueous extracts from non-woody plants of the genus Hesperaloe prepared according to the present invention may comprise from about 2 to about 10 wt % saccharides, from about 10 to about 25 wt % saponin, from about 0 to about 5 wt % protein, from about 0 to about 5 wt % lipids and from about 20 to about 30 wt % inorganic salts, where the weight percentages are based upon the total weight water soluble solids.

Generally milling is carried out with the addition of an aqueous solvent, such as water, having a pH ranging from about 5 to about 9, such as from about 6 to about 7 to about 8. As such, milling is generally carried out without the addition of chemicals that would solubilize cellulose or lignin, such as sodium hydroxide (NaOH), sodium carbonate (Na₂CO₃), sodium bicarbonate (NaHCO₃) potassium hydroxide (KOH), potassium carbonate (K₂CO₃), potassium bicarbonate (KHCO₃), ammonium hydroxide (NH₄OH) or combinations thereof. Rather, the milling process may simply be carried out with water or with juice extracted from the biomass during the milling process. The water soluble solids are generally recovered from the milling process as a crude extract and may be subjected to further processing to recover specific compounds, such as saccharides, polysaccharides, organic acids and saponins.

The suspended solids, also referred to herein as the water insoluble fraction, may be removed from the crude extract by well-known processes including, for example, clarification, filtration, centrifugation, or a combination thereof. The amount of water insoluble solids in the extract (on bone dry grams per kilogram of bone dry biomass) may range from about 1.0 to about 30 grams and may comprise hydrophobic substances such as waxes and the like.

After removal of suspended solids, the clarified juice may be used directly, concentrated, or subjected to further processing to isolate one or more water soluble solids such as saccharides, polysaccharides, organic acids, saponins and sapogenins. In other instances, the clarified juice may be further purified to remove saccharides, polysaccharides, and organic acids to yield composition comprising saponins.

In a particularly preferred embodiment, the invention provides a process for removing water soluble solids from non-woody plants of the genus Hesperaloe by passing the biomass through a series of mills, such as two, three, four, five, six or seven mills. These mills may comprise horizontal cylinders or rolls arranged in groups of three with a first roll disposed above a pair of horizontally aligned rolls in a triangle formation. In certain instances, the bottom two rolls may be fixed, and the top roll is rotated by a motor. The diameter of the rolls may range from about 50 to about 100 cm and they may have a length from about 1 to about 3 m. In certain preferred embodiments the rolls have plurality grooves disposed on their outer most surface and extending continuously about the circumference of the roll. The grooves may have a rectangular cross-section with depths ranging from about 2 to about 5 cm.

The grooves in the cylinders or rolls are found to create openings in the outer epidermal layer of the biomass and improve wash fluid penetration. Further, milling the biomass with grooved cylinders or rolls facilitates removal of the epidermal layer, which may improve subsequent pulping of the biomass. Separation of the epidermal layer may also facilitate removal of the pith, which may also improve subsequent pulping of the biomass. Removal of the pith may also be facilitated by the inclusion of one or more depithers interspersed between mills.

Rolls within a mill may be loaded against one another to create a nip therebetween. In those embodiments where the rolls are arranged in a triangle formation, the upper most roll may be loaded against the bottom rolls by a applying a loading pressure to the upper most roll. The rolls turn at about 5-50 rpm, and the velocity of the biomass through the rolls is about 10-25 cm/second.

After passing through each mill, the biomass, which may at this point in the process be referred to generally as bagasse, is transported to the next mill, such as by a conveyor. In order achieve the desired degree of extraction, a countercurrent system of liquid spray is employed. For example, in a multi-mill extraction process the biomass going to the final mill is sprayed with an aqueous solvent, such as heated water, to remove water soluble solids from the bagasse. The resultant extract, sometimes called a crude extract or a juice, is then sprayed on the bagasse prior to entering the next to last mill, and so on to the first mill. As a result, the amount of water soluble extracts in the countercurrent liquid stream is increasing each time it is sprayed onto the biomass. The juice may be subjected to further processing to provide the desired end product.

In certain instances, the process may also comprise one or more diffusers in addition to one or more mills. In a diffuser the biomass travels countercurrent to a liquid, such as water having a temperature from about 25 to about 95° C. Suitable diffusers include a ring diffuser, a tower diffuser, or a drum diffuser. Exemplary diffusion systems are discussed, for example, in U.S. Pat. Nos. 4,182,632, 4,751,060, 5,885,539 and 6,193,805 the contents of which are hereby incorporated in a manner consistent with the present disclosure. Numerous other diffusion methods and devices for the diffusion method are known and can be adapted for use in the methods described herein. One such diffuser is the continuous-loop, counter-current, shallow-bed Crown Model III Percolation Extractor, commercially available from Crown Iron Works, Blaine, Minn.

In other instances, the process may comprise one or more depithers. The depither may be a vertical depither and consists of a rotor assembly which holds an array of blades and a perforated cylinder. This cylinder is also sometimes called the screen. The rotor assembly rotates within the cylinder with a clearance of about 1 to 2 cm between the cylinder and the ends of the blades of the rotor assembly. The rotor assembly is either directly driven by an electric motor or indirectly through a chain or gears. The rotor assembly will rotate at about 1500 to 3000 revolutions per minute (rpm) in use and consequently must be well balanced. The biomass being processed enter at the top of the cylinder and are processed by the blades on the rotor assembly as they move downward. The pith and epidermal debris are forced out of the area of the rotor assembly through the perforations in the cylinder. The fiber and the removed pith are then separately collected.

Suitable depithers include, for example, those described in U.S. Pat. Nos. 3,537,142 and 4,641,792, which are incorporated herein by reference in a manner consistent with the present invention. Preferably, a depither is interspersed in the extraction process between two of the extraction mills. Biomass is fed from an extraction mill to the depither with the biomass output from the depither flowed to a subsequent extraction mill. Liquids from the depither may be fed to a prior extraction mill although they could be fed to a subsequent extraction mill.

In a particularly preferred embodiment, the depither has a countercurrent liquid flow. Fresh wash liquid may be injected into the lowermost section with the liquid stream exiting from the lowermost section being injected into the middle section. The wash liquid exiting the middle section of the depither may in-turn be injected into the uppermost section. Liquid exiting the uppermost section of the depither may be flowed to the prior extraction mill and used as the wash water in the preceding extraction step.

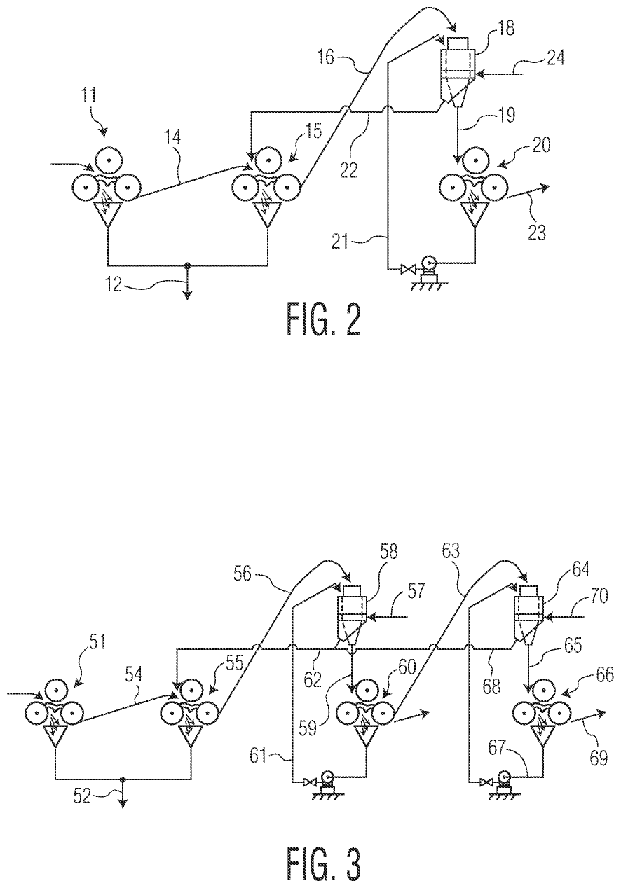

With reference now to drawings, a three-stage extraction mill with a single depither is illustrated. The biomass is prepared before being flowed to the first extraction mill. Preparation may comprise cutting the biomass to a given length, such as from about 0.25 to about 50 cm, such as from about 0.5 to about 25 cm. The biomass may also be cleaned prior to extraction to remove soil and other debris. The biomass is then put onto tables for feeding to the extraction mills. In FIG. 2 there is shown the feeding of the biomass to the first extraction mill 11, the liquids extracted from the biomass are collected 12 and the biomass 14 passes to the second extraction mill 15. The liquids from second extraction mill 15 are likewise collected 12 with the biomass 16 passing to a first depither 18. The biomass 19 exits the depither and flows to a third extraction mill 20. The liquids 21 extracted in the third extraction mill 20 are flowed to the biomass input to the depither 18 to wet the biomass 16 as it enters the depither 18. A second liquid 24, which may be fresh water, enters the middle section of the depither. The liquids 22 from the depither 18 are flowed to the second extraction mill 15. The pressed and extracted biomass 23 flows from the third extraction mill 20 and may be subjected to further processing.

FIG. 3 shows a system comprised of four extraction mills 51, 55, 60, 66 and two depithers 58, 64. The biomass is prepared and flowed to the first extraction mill 51. The processed biomass 54 exits the first extraction mill 51 and is fed to the second extraction mill 55. The liquid 52 from the second extraction mill 55 is collected with the biomass 56 passing to the first depither 58. The biomass 59 exits this first depither and flows to third extraction mill 60. The liquid 61 from this extraction mill flows to the biomass input to depither 58. The liquid stream 62 from this depither flows to the biomass input of the third extraction mill. A biomass 63 exits this extraction mill 60 and flows to second depither 64. A biomass 65 exits this depither and passes to the fourth extraction mill 66. A biomass 69 exits extraction mill 66, with a liquid stream 67 flowing to wet the input biomass to depither 64. A liquid stream 68 from depither 64 flows to the second extraction mill. Fresh liquid 70 is flowed to depither 64.

In particular instances, saponins may be extracted and recovered from non-woody plants of the genus Hesperaloe according to the present invention. As used here, the term saponin generally refers to a compound consisting of a triterpenoid of oleanane structure and one or more glycosides, the glycosides being bound to the triterpenoid at the 3 position and/or at the 28 position. The term glycoside is intended to mean all sugars including glucose found naturally in non-woody plants of the genus Hesperaloe including arabinose, glucose, galactose, xylose, and glucuronic acid.

Saponins may be obtained by sequentially extracting the biomass from non-woody plants of the genus Hesperaloe with water and then further treating the water soluble fraction with a water-immiscible polar solvent to form a polar solvent-saponin mixture. Suitable water-immiscible polar solvents include, for example, alcohols having from 4 to 6 carbon atoms, such as butyl, amyl, hexyl and cyclohexyl alcohols. The polar solvent may be removed from the saponin-containing mixture to produce a saponin-containing product.

The juice resulting from the foregoing extraction process may be subjected to further extraction to obtain saponin in the form of a crude saponin extract or its substantially purified form comprising saponins at a concentration from about 30 to about 90% in weight. The extraction method may comprise mixing juice extracted from non-woody plants of the genus Hesperaloe with a water-immiscible polar solvent. Suitable water-immiscible polar solvents include, for example, alcohols having from 4 to 6 carbon atoms, such as butyl, amyl, hexyl and cyclohexyl alcohols. Extraction of the juice with a water-immiscible polar solvent generally removes impurities such as proteins, carbohydrates, and organic acids, which remain in the aqueous phase, the saponin being transferred to the solvent phase.

The solvent phase containing the saponin may be subjected to further treatment to separate the saponin from the alcohol phase. This can be accomplished in various ways including, for example, by cooling, by dehydrating the solvent extract, or by adding an organic solvent which is miscible with the alcohol solvent but in which the saponin is insoluble. Suitable precipitating solvents include, for example, diethyl ether, petroleum ether, acetone, and chloroform.

In a particularly preferred embodiments, the saponin is separated from the alcohol by flash evaporation. Flash evaporation is a technique known in preparative chemistry for the rapid removal of a volatile component from a liquid mixture. The volatile liquid is removed from solution by rapid conversion to a vapor phase by creating a thin film of the solution over a large surface area under reduced pressure often accompanied by an increase of temperature of the solution above ambient but less than the boiling point of the solution at atmospheric pressure. The actual thickness of the film and the area over which it is applied is chosen to provide optimum evaporation and ease of use, but evaporation may be substantially instantaneous (hence the name “flash” evaporation). Flash evaporation avoids the prolonged use of high temperatures that may degrade the intended product and has the ability to remove almost all of the alcohol component (which makes the remaining solution suitable for the preferred practice of spray drying employed in the next step. The alcohol may be recovered from this step and reused in the extraction process.

The saponin content of the alcohol extract can be further increased by passage over an ultrafiltration membrane without significant alteration to or loss of the saponin composition. This concentrated saponin fraction where the saponin content is in the range of 85-90%, can then be further purified in a liquid state or reduced to a dry state. Individual saponins may be recovered by a combination of reversed-phase solid phase extraction and preparative reversed-phase HPLC. Alternatively, the alcohol extract containing saponins can be fractionated directly by a combination of reversed-phase solid phase extraction and preparative reversed-phase HPLC.

In still other embodiments saponins may be purified from juice prepared according to the present invention comprises the steps of mixing the juice with a salt and a solvent to form a first solution. The solvent may comprise one or more solvents selected from acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme, 1,2-dimethoxyethane, dimethylformamide, dimethylsulfoxide, 1,4-dioxane, ethanol, ethyl acetate, ethylene glycol, glycerin, heptane, hexamethylphosphoramide, hexamethylphosphorous triamide, hexane, methanol, methyl-t-butyl ether, methylene chloride, N-methyl-2-pyrrolidinone, pentane, perchloroethylene, petroleum ether, 1-propanol, 2-propanol, pyridine, tetrahydrofuran, toluene, triethylamine, trifluorotoluene, water, xylene, or any combination of the forgoing. In some embodiments the solvent is water. The salt may be selected from an alkali metal salt, an alkaline earth salt, a transition metal salt, an ammonium salt, or combinations of the forgoing. In certain preferred embodiment the salt added to the plant extract to form the solution is an alkaline earth metal salt. In particularly preferred embodiments the salt is calcium chloride (CaCl₂), magnesium chloride (MgCl₂), or a mixture thereof.

The pH of the first solution is generally adjusted to a pH from about 6.0 to about 9.0, such as from about 6.0 to about 8.0, such as from about 6.0 to about 7.0. At least one phosphate may then be added to the first solution to form an ion-polysaccharides complex precipitate. Useful phosphates include, for example, sodium hydrogen phosphate (Na₂HPO₄), sodium dihydrogen phosphate (NaH₂PO₄), sodium phosphate (Na₃PO₄), or sodium hydrogen bisphosphate (Na₂H₂PO₇).

The precipitated ion-polysaccharides complex may be removed by filtration to yield a second solution, which may be further clarified to produce an extract of purified saponins. Optionally, the extract can be concentrated by any filtration technique known in the prior art. Preferably, the concentration of the extract of purified saponins is carried out by nanofiltration, ultrafiltration and diafiltration, or any combination of these techniques. In some embodiments, the saponin extract is substantially free of proteins. In some embodiments, the saponin extract is substantially free of polysaccharides. In some embodiments, the saponin extract is substantially free of phenolic compounds.

In other instances, the crude or partially purified saponin may be acidified to produce sapogenins. First a solution of saponins in an alcohol is prepared as described herein and then a strong acid, preferably 1-3.5 N, is added to the solution to hydrolyze the saponins to form corresponding sapogenins. The sapogenins may be further processed by precipitation, recovering the precipitate, and decolorizing the precipitate by forming a slurry of the precipitate with a solution of an aqueous base to form a decolorized sapogenin product.

In a particularly preferred embodiment sapogenins can be obtained by acid hydrolysis of a solution of saponins in an alcohol, for example using 450 mL concentrated HCl per 3 L of the alcohol extract under reflux. The hydrolysate is allowed to cool resulting in the formation of a precipitate which is recovered by filtration. The precipitate is slurried in water and the resulting slurry is adjusted preferably to pH 10 with a base. The sapogenins precipitate from the basic solution as off-white crystals and are recovered by filtration. The resulting crystalline precipitate may be washed with dilute acid and distilled water until the effluent is clear. The precipitate containing the sapogenins may then be air-dried and can be further refined by recrystallization.

The individual sapogenins may be recovered from this mixture, e.g. by preparative HPLC using reversed-phase adsorbents. The purification can also be achieved on a large scale by selective desorption from a reversed-phase solid-phase extraction cartridge eluted with a step gradient of aqueous methanol. Preparative HPLC and systems such as simulated moving bed chromatography are frequently in commercial use for recovery of high value solutes from solutions. The sapogenins may be further purified by recrystallization from hot 95% alcohol.

The saponin content of the various fractions during extraction may be monitored, for example, by HPLC analysis of a filtered 50% (v/v) ethanol or methanol extraction by chromatography on C-8 or C-18 RP columns eluted with a 0.05% Trifluoroacetic acid (v/v) (TFA) in water:methanol gradient, or a 0.05% TFA in water:acetonitrile gradient. Saponins in the samples may be detected by Evaporative Light Scattering Detection (ELSD) using, for example, Model PL-EMD 960 from Polymer Laboratories. Acetic acid (1%) can be used in place of TFA and chromatographic separation can be achieved by isocratic elution. The sapogenin content of extracts and samples derived by hydrolysis can also be determined using the same chromatographic procedure.

The total saponin content in Hesperaloe, on the basis of grams of total saponin per kilogram of bone dry biomass, may range from about 50.0 to about 150 g/kg, such as from about 5.0 to about 120 g/kg, such as from about 80.0 to about 120 g/kg. In other instances, water soluble solids prepared from Hesperaloe biomass according to the present invention may comprise at least about 5 wt % of total saponins, such as from about 5 to about 15 wt %, such as from about 8 to about 12 wt %, based upon the dry weight of the water soluble solids. The saponins may be provided as part of a crude juice, as part of a dried water soluble solids compositions, as a partially purified compositions or as a substantially pure composition comprising a mixture of saponins.

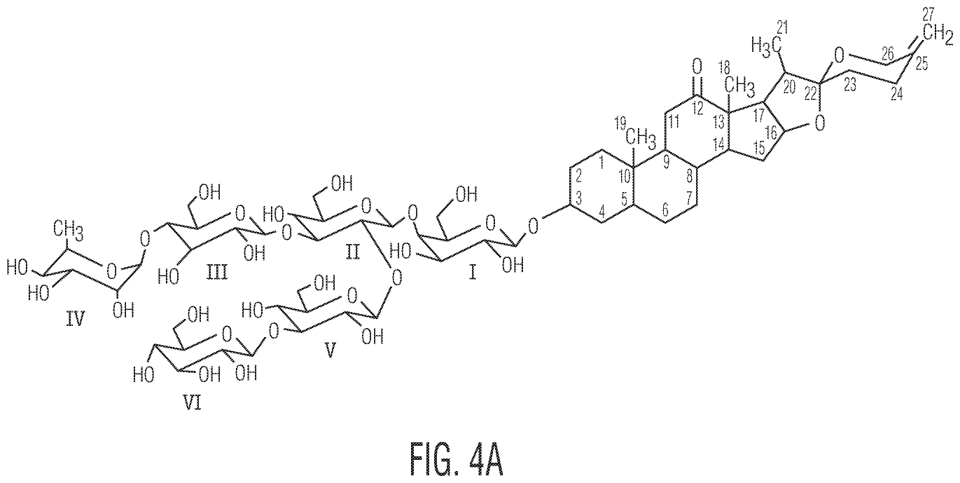

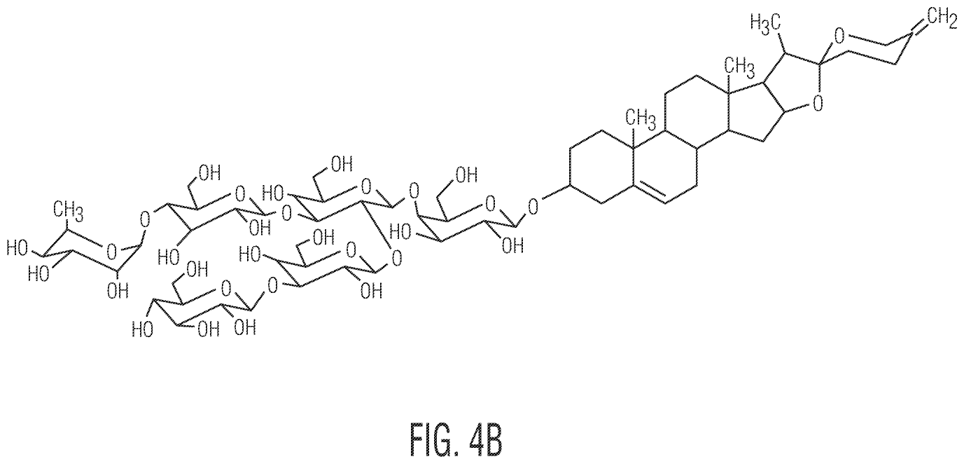

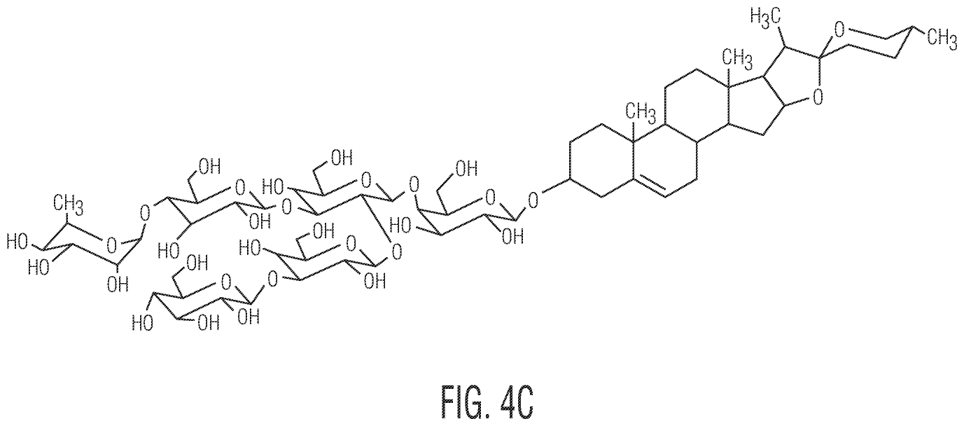

In certain embodiments saponins extracted from Hesperaloe biomass may have at least one of the following aglycones or genins: kammogenin, manogenin, gentrogenin, hecogenin, tigogenin, sarsapogenin, chlorogenin and gitogenin or their corresponding isomer or oxidized or reduced forms with at least one of the following glycosidic moieties (in the form of acid or salt): glucose, xylose, rhamnose, arabinose, or galactose. In other embodiments the saponins may comprise agamenoside, agaveside, agavoside, magueyside, agavasaponi, cantalasaponin, sisalsaponin, gabrittonoside, dongnoside or amolonin, or other steroidal saponins. In certain instances, the saponins may comprise 25(27)-dehydrofucreastatin (FIG. 4A), 5(6),25(27)-disdehydroyuccaloiside C (FIG. 4B), 5(6)-disdehydroyuccaloiside C (FIG. 4C), furcreastatin and yuccaloiside C.

In still other embodiments, water soluble saccharides and polysaccharides may be extracted and recovered from non-woody plants of the genus Hesperaloe. For example, biomass may be milled and extracted as described above to produce a first aqueous solution containing inulin and impurities including at least one member of the group consisting of minerals, amino acids, proteins, fats, cell wall fragments, colloidal matter, and particulate matter such as dirt and, subjecting said first aqueous solution to a denaturing step (e.g., by heating) to denature at least one enzyme selected from the group consisting of inulin degrading enzymes to produce a second aqueous solution.

The second aqueous solution may be clarified by one of the methods taught in the prior art such as, for example, centrifugation, filtration, filtration with the aid of diatomaceous or siliceous earths, carbon treatment, or cross-flow membrane filtration. The preferred filtration method is press filtration utilizing a filter having a 1 to 20 micron nominal pore dimension, most preferably a 5 to 10 micron nominal pore dimension. Filtration of the more finely suspended solids may be improved by the addition of diatomaceous earth. Preferably clarification removes particulate matter, colloidal matter, colored impurities, or microorganisms to produce a third aqueous solution.

In other embodiments suspended solids may be removed from the second aqueous solution by thermal coagulation. More particularly, the second aqueous solution shaken while heating to approximately 50 to 90° C., and most preferably 70 to 80° C. with the addition of diatomaceous earth during which time colloids coagulate. The coagulants may then be removed by either centrifugation or filtration to produce a partially purified inulin extract or third aqueous solution.

The third aqueous solution may be subjected to further treatment by passing the solution over an absorbent medium such as activated carbon, or adsorbent resins, or a combination of both, to remove ionic impurities and color-forming impurities and yield a fourth aqueous solution. For example, the third aqueous solution may be further purified with cationic resins such as bead-form, strong acid, gel-type cation exchange resins based on crosslinked polystyrene with sulfonic acid function groups to produce an acidified, demineralized inulin extract. The extract may have a pH from 1.8 to 2.3. The cationic exchange may be conducted at about 85° C.

The acidified, demineralized inulin extract may then be subjected to an anionic resin such as a bead-form, highly basic anion exchange resin having a structure based on crosslinked polystyrene with quaternary ammonium functional groups. The eluent is a hydrolyzed and demineralized inulin extract in which the molecular weight of the inulin and other carbohydrates in the extract are substantially reduced.

The purified inulin solution may be further treated by filtration to separate inulin having different average degrees of polymerization. For example, purified inulin solution may be passed through an ultrafiltration membrane having a predetermined pore size whereby inulin fractions having average degrees of polymerization less than a predetermined value pass through said membrane as permeate and inulin fractions having average degrees of polymerization greater than said predetermined value are collected as retentate.

Inulin having the desired degree of polymerization may be dried by any method known to those skilled in the art including, for example, precipitation, crystallization, spray-drying, drum drying, and the like.

### Test Methods

### Water Soluble Solids

Total biomass water soluble solids may be determined using an Accelerated Solvent Extraction system (ASE) such as a Dionex™ ASE™ 350 (Thermo Fisher Scientific, Waltham, Mass.). Approximately grams of harvested biomass is dried to a constant weight in an oven, typically 4 hours at 125° C. After drying 1.5-2.0 grams of the bone dry biomass is accurately weighed and the weight (W_{b}) recorded to the nearest 0.001 gram. Using water as the solvent, biomass is extracted using the conditions set forth in the table below. The ratio of biomass to solvent is generally 21:1 and five consecutive water extraction cycles are performed. At the end of each extraction cycle, the liquid phase is collected, dried under vacuum at approximately 40° C. and the weight of the dried material (Wᵢ) is recorded to the nearest 0.001 g. The total weight of water soluble solids (Wₑ) is calculated by summing the weight of solids recovered from each extraction cycle (Wᵢ). Total water soluble solids as a percentage of bone dry biomass is then determined using the following equation: Water Soluble Solids (wt %)=Wₑ/W_{b}*100.

| | |
|---|---|
| Pressure (psi) | 1500 |
| Temperature (° C.) | 40 |
| Static Time (min.) | 10 |
| Cycles (no.) | 5 |

The total water soluble solids in biomass extract may be determined by withdrawing an appropriate aliquot, typically about 10-50 ml, transferring to clean, dry, centrifuge tube. The tube is centrifuged at 7000 rpm for 20 minutes. The weight of extract (W₁) is calculated. An aliquot of the supernatant is then transferred to clean, pre-weighed beaker (D₀), and weighed. The beaker and sample are then weighed to the nearest 0.001 g and the weight (D₂) recorded. The beaker containing the sample is then placed at 140° C. in a hot air oven for overnight drying. The beaker is removed from the oven and desiccated to cool to room temperature then weighed to the nearest 0.001 gram (D₁). The weight percentage of soluble solids, based upon the weight of the extract, is determined using the formula below:

$$Water Soluble Solids ( wt \% ) = \frac{( {D}_{1} - {D}_{0} ) \times 100}{( {D}_{2} - {D}_{0} )}$$

D₁=mass of empty beaker+dried soluble solids, D₀=mass of empty beaker, D₂=mass of biomass extract and empty beaker.

### Total Saponins

Total saponins were measured generally as described in Makkar, Harinder P. S., Sidhuraju, P., Becker, Klaus (2007) Plant Secondary Metabolites, chapter 17, pp 93-100. A standard saponin solution was prepared by weighing 10 mg of diosgenin (MilliporeSigma >93%), dissolving in 16 mL of methanol and adding 4 mL of distilled water. The solution was mixed thoroughly to yield a 0.5 mg/mL diosgenin solution in 80% methanol solvent. The standard was used to produce a calibration curve by transferring various amounts of the standard (0, 10, 20, 40, 60, 80, and 100 μL) into 13-mm glass test tubes. A solution of 80% aqueous methanol was added to a total volume of 100 μL.

Prior to testing samples of biomass extract were adjusted to about 0.5 wt % total solids by dilution with water to ensure absorbency result fell along the saponin standard calibration curve range. Samples of diluted extract (20-μL) were pipetted into 13-mm glass test tubes and the volume was brought up to 100 μL with 80 μL methanol. Each sample was tested in triplicate.

To each sample 100 μL of vanillin reagent (prepared by dissolving 800 mg of vanillin in 10 mL of 99.5% ethanol (analytical grade)) and then 1.0 mL of 72% (v/v) sulfuric acid (72% (v/v) sulfuric acid prepared by adding 72 mL of sulfuric acid (analytical grade, 95%, w/w) to 28 mL of distilled water) were added. Solutions were mixed well and heated at 60° C. for 10 minutes. Samples were then cooled in an ice bath and 1 mL of solution was transferred into respective cuvette and absorbance at 544 nm was read. The total mass of saponins in the sample may be calculated based upon the standard absorbency curve as follows:

Saponin (μg)=[Slope]×Measured Absorbency−[Intercept]

### EXAMPLES

### Water Soluble Extractives

The inventive extract was prepared by forage harvesting mature Hesperaloe funifera leaves above the crown, cutting the leaves into pieces ranging from about 0.50 to about 8.0 cm and pressing the cut biomass using a sugar cane tandem press, each mill of the tandem press having 3-rollers. The biomass was passed through the tandem mill three times. Imbibition water was added prior to first mill in the tandem. The crude juice was collected and passed through 25 mm filter and heated to boiling in a flat pan evaporator (Leader Evaporator Company, Swanton, Vt.) to concentrate the extract to 29% solids. The water-soluble solids comprised 21 wt % total saponins, based upon the bone dry weight of water soluble solids. The composition of the water soluble solids is further described in Table 2, below.

TABLE 2

| | Weight Percent of |
|---|---|
| | Water Soluble Solids (wt %) |
| Saponins | 20 |
| Saccharides | 16 |
| Minerals | 24 |
| Fats | 5 |
| Protein | 11 |

Extraction and Purification of Saponins from Hesperaloe

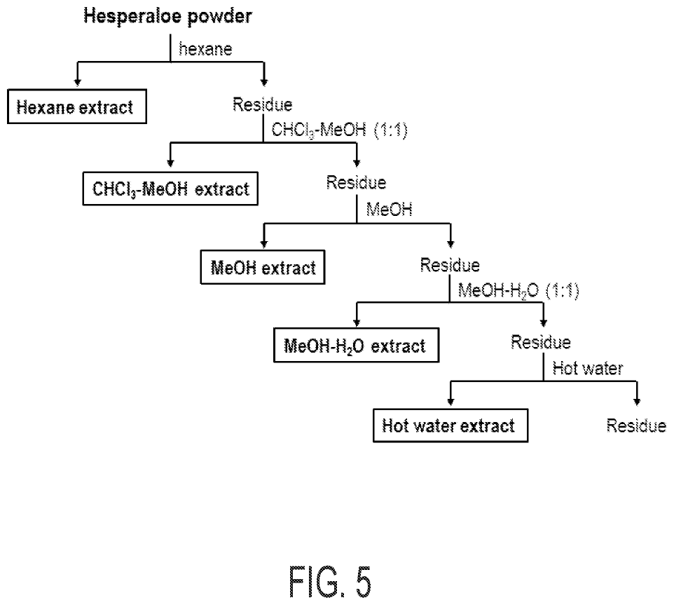

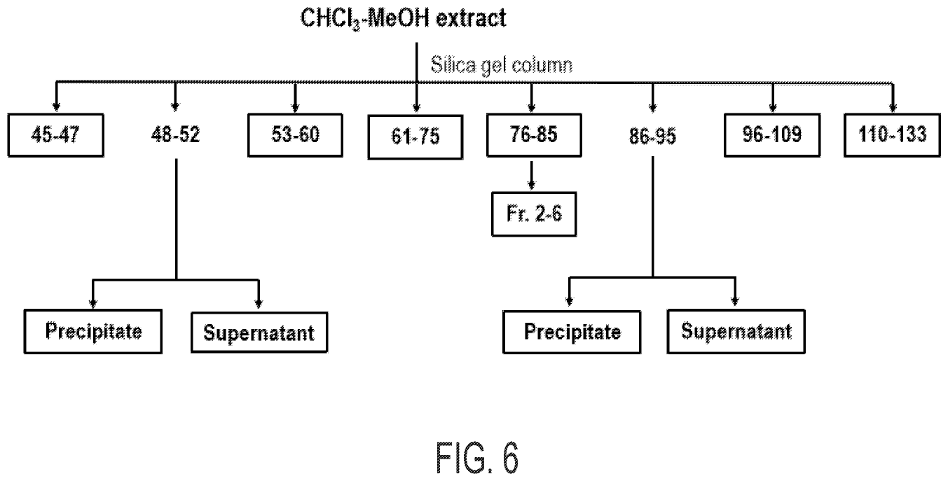

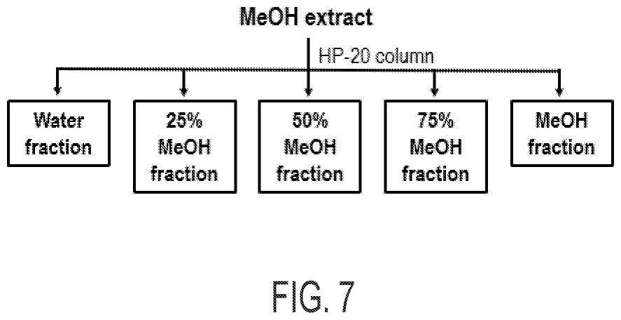

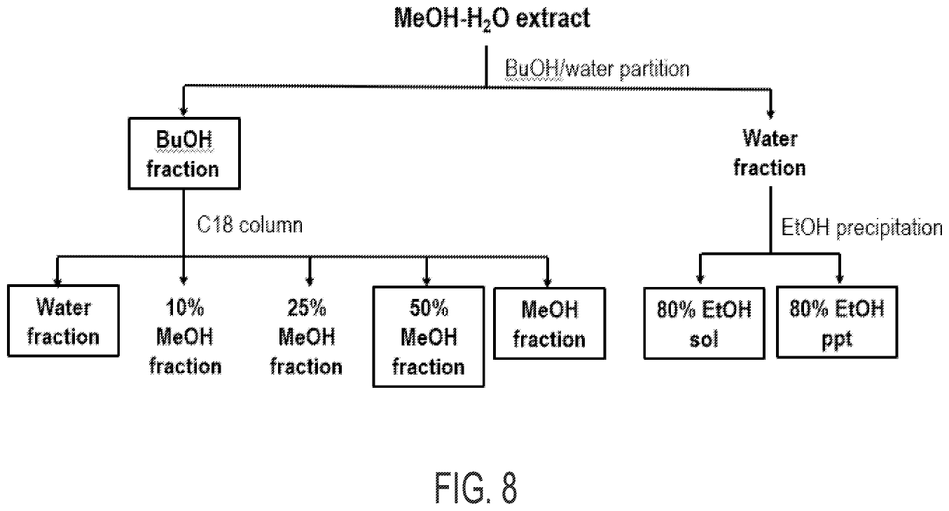

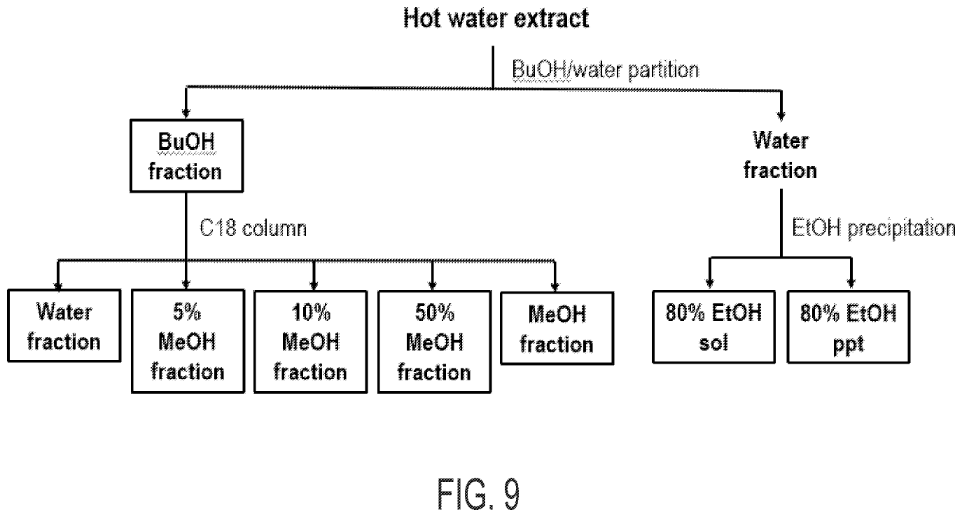

Five-hundred (500) grams of dry, coarsely ground Hesperaloe funifera was sequentially extracted by soaking (15 h) biomass with occasional stirring using Hexane (H), 1:1 Chloroform/Methanol (CM), Methanol (M), Methanol/Water (MW) and Hot Water (HW) to yield a crude extract. The extraction scheme is illustrated in FIGS. 5-9 and further described below. Extraction conditions and yields are provided in Table 3, below. All extracts were reduced in volume using rotary evaporator and freeze dried.

TABLE 3

| | Amount | Conditions | Yield | Yield |
|---|---|---|---|---|
| Solvent | (L) | (° C.) | (g) | (%) |
| Hexanes | 20 | RT | 3.18 | 0.64 |
| CHCl₃—MeOH (1:1) | 20 | 30-40 | 36.76 | 7.35 |
| MeOH | 21 | 30-40 | 25.08 | 5.02 |
| MeOH—H₂O (1:1) | 20 | 78-80 | 32.03 | 6.41 |
| H₂O | 20 | 78-80 | 24.27 | 4.85 |

The extracts were subjected to partitioning and purification using high performance liquid chromatography/mass spectrometry (HPLC/MS), which yielded 30 fractions, summarized in Table 4.

TABLE 4

| Sample # | Sample ID | Sample Description |
|---|---|---|
| 1 | H | Hexane extract |
| 2 | CM | CHCl₂—MeOH (1:1) extract |
| 3 | M | MeOH extract |
| 4 | MW | MeOH—H₂O (1:1) extract |
| 5 | HW | Hot water extract |
| 6 | CM F45-47 | Fraction 45-47 from CHCl₂—MeOH (1:1) extract |
| 7 | CM F48-52 ppt | Fraction 48-52 precipitate from CHCl₂—MeOH (1:1) extract |
| 8 | CM F48-52 sup | Fraction 48-52 supernatant from CHCl₂—MeOH (1:1) extract |
| 9 | CM F53-60 | Fraction 53-60 from CHCl₂—MeOH (1:1) extract |
| 10 | CM F61-75 | Fraction 61-75 from CHCl₂—MeOH (1:1) extract |
| 11 | CM F76-85 | Fraction 76-85 from CHCl₂—MeOH (1:1) extract |
| 12 | CM F86-95 ppt | Fraction 86-95 precipitate from CHCl₂—MeOH (1:1) extract |
| 13 | CM F86-95 sup | Fraction 86-95 supernatant from CHCl₂—MeOH (1:1) extract |
| 14 | CM F96-109 | Fraction 96-109 from CHCl₂—MeOH (1:1) extract |
| 15 | CM F110-133 | Fraction 110-133 from CHCl₂—MeOH (1:1) extract |
| 16 | CM F76-85 F2-6 | Fraction 2-6 from Fraction 76-85 from CHCl₂—MeOH (1:1) extract |
| 17 | M-HP20-W | Water fraction from MeOH extract |
| 18 | M-HP20-25M | 25% MeOH fraction from MeOH extract |
| 19 | M-HP20-50M | 50% MeOH fraction from MeOH extract |
| 20 | M-HP20-75M | 75% MeOH fraction from MeOH extract |
| 21 | M-HP20-M | MeOH fraction from MeOH extract |
| 22 | MW-B | n-BuOH fraction from MeOH—H₂O (1:1) extract |
| 23 | MW-80E sol | 80% EtOH soluble fraction from MeOH—H₂O (1:1) extract |
| 24 | MW-80E ppt | 80% EtOH precipitate from MeOH—H₂O (1:1) extract |
| 25 | MW-B-C18-W | Water fraction from BuOH fraction from MeOH—H₂O (1:1) extract |
| 26 | MW-B-C18-50M | 50% MeOH fraction from BuOH fraction from MeOH—H₂O (1:1) extract |
| 27 | MW-B-C18-M | MeOH fraction from BuOH fraction from MeOH—H₂O (1:1) extract |
| 28 | HW-B | n-BuOH fraction from hot water extract |
| 29 | HW-80E sol | 80% EtOH soluble fraction from hot water extract |
| 30 | HW-80E ppt | 80% EtOH precipitate from hot water extract |
| 31 | HW-B-C18-W | Water fraction from BuOH fraction from hot water extract |
| 32 | HW-B-C18-5M | 5% MeOH fraction from BuOH fraction from hot water extract |
| 33 | HW-B-C18-10M | 10% MeOH fraction from BuOH fraction from hot water extract |
| 34 | HW-B-C18-50M | 50% MeOH fraction from BuOH fraction from hot water extract |
| 35 | HW-B-C18-M | MeOH fraction from BuOH fraction from hot water extract |

### Fraction of CHCl₃-MeOH (1:1) Extract

Fractionation of the CHCl₃-MeOH (1:1) extract was performed by normal phase chromatography using a CombiFlash system. Sample of 6 g was dissolved in CHCl₃, pre-absorbed on Si gel and loaded onto prepacked 330 g Si column preconditioned with CHCl₃ (solvent A). The column was eluted with a gradient of 0-100% MeOH with 0.5% H₂O (solvent B). The fractions were combined based on their TLC profile, yielding the following pooled fractions: Fr. 14-44, 45-47, 48-52, 53-60, 61-75, 76-85, 86-95, 96-109, and 110-133.

### Fraction of MeOH Extract

Fractionation of the MeOH extract was carried on a HP-20 column due to solubility issue. MeOH extract (10 g) dissolved in MeOH/H₂O was pre-absorbed on Diaion HP-20 resin (250 mL) and the solvent was removed. It was then loaded on the HP-20 column (2 L Buchner funnel containing 1 L of HP-20 resin preconditioned with dH₂O). The column was first eluted with 2 L dH₂O, then with the same volume of H₂O/MeOH in different concentrations to yield five fractions, namely M-HP20-W, -25M, -50M, -75M, and -M.

### Fraction of the MeOH—H₂O (1:1) and Hot Water Extracts

These two extracts were first fractioned by partitioning (liquid-liquid extraction) with butanol, followed by ethanol precipitation. The dry extract samples were dissolved in water and repeatedly extracted with equal volumes of n-BuOH pre-saturated with water. The n-BuOH soluble fraction was concentrated on the rotary evaporator and freeze dried (MW-B and HW-B).

TABLE 5

| | | LC-MS | |
|---|---|---|---|
| Molecular | | Retention time | Structure |
| formula | Fraction ID | (RT) | info |
| C₃₂H₅₂O₈ | CM F48-52 sup F27-28 | 2.36 | 1 sugar |
| C₃₃H₅₂O₉ | CM F48-52 sup F29-31 | 2.94 | 1 sugar |
| C₃₃H₅₆O₉ | CM F48-52 sup F27-28 | 2.50 | 1 sugar |
| C₃₉H₆₂O₁₃ | CM F61-75 F28-30 | 2.81 | 2 sugars |
| | CM F61-75 F31 | 2.84 | |
| C₃₉H₆₂O₁₃ | CM F61-75 F32-34 | 3.03 | 2 sugars |
| C₃₉H₆₂O₁₄ | CM F76-85 F27-29 | 2.52 | 2 sugars |
| C₃₉H₆₂O₁₄ | CM F61-75 F28-30 | 2.60 | 2 sugars |
| | CM F61-75 F31 | 2.62 | |
| | CM F76-85 F27-29 | 2.62 | |
| | CM F76-85 F30-31 | 2.61 | |
| C₃₉H₆₂O₁₄ | CM F61-75 F31 | 2.84 | 2 sugars |
| C₃₉H₆₄O₁₅ | CM F76-85 F21-23 | 1.85 | 2 sugars |
| C₄₅H₇₀O₁₇ | CM F61-75 F32-34 | 3.03 | 3 sugars |
| C₄₅H₇₂O₁₇ | CM F61-75 F32-34 | 3.11 | 3 sugars |
| C₄₅H₇₂O₁₇ | CM F76-85 F30-31 | 2.85 | 3 sugars |
| C₄₅H₇₂O₁₈ | CM F76-85 F30-31 | 2.73 | 3 sugars |
| C₅₁H₈₀O₂₂ | CM F76-85 F30-31 | 2.93 | 4 sugars |
| C₅₁H₈₂O₂₂ | CM F76-85 F30-31 | 3.01 | 4 sugars |
| | CM F76-85 F32-33 | 3.03 | |
| C63H₁₀₀O₃₂ | CM F86-95 ppt RS12-97-5 | 2.89 | 6 sugars |
| C₆₃H₁₀₀O₃₃ | CM F86-95 ppt RS12-97-2 | 2.57 | 6 sugars |
| C₆₃H₁₀₂O₃₂ | CM F86-95 ppt RS12-97-6 | 2.95 | 6 sugars |
| | CM F96-109 F20-24 | 2.95 | |
| C₆₃H₁₀₂O₃₃ | CM F86-95 ppt RS12-97-4 | 2.61 | 6 sugars |
| | CM F96-109 F15-19 | 2.62 | |
| | M-HP20-M F40 ppt | 2.58 | |
| | MW-B-C18-M F14 ppt | 2.59 | |
| C₆₃H₁₀₂O₃₃ | M-HP20-M F40 ppt | 2.76 | 6 sugars |
| | MW-B-C18-M F14 ppt | 2.80 | |
| C₆₃H₁₀₄O₃₂ | CM F86-95 ppt RS12-97-6 | 3.01 | 6 sugars |
| | CM F96-109 F20-24 | 3.00 | |
| | M-HP20-M F40 ppt | 2.96 | |
| | MW-B-C18-M F14 ppt | 2.99 | |
| C₆₃H₁₀₄O₃₂ | M-HP20-M F40 ppt | 3.14 | 6 sugars |

The novel saponins, 25(27)-dehydrofucreastatin (FIG. 4A), 5(6),25(27)-disdehydroyuccaloiside C (FIG. 4B), and 5(6)-disdehydroyuccaloiside C (FIG. 4C) were identified in fractions CM F86-95 ppt RS12-97-5, CM F86-95 ppt RS12-97-2 and CM F86-95 ppt RS12-97-6, respectively. The saponins furcreastatin and yuccaloiside were also identified.

1. A composition extracted from a non-woody plant of the genus Hesperaloe, the extract comprising 25(27)-dehydrofucreastatin, 5(6),25(27)-disdehydroyuccaloiside C, 5(6)-disdehydroyuccaloiside C, furcreastatin, yuccaloiside C, or a mixture thereof.

2. The composition of claim 1 wherein the extract is water soluble.

3. The composition of claim 1 comprising at least about 5 wt % saponins, based upon the bone dry weight of the composition.

4. The composition of claim 1 comprising from about 5 to about 40 wt % saponins, based upon the bone dry weight of the composition.

5. The composition of claim 1 further comprising inorganic salts, saccharides, lipids, or proteins.

6. The composition of claim 1 comprising 25(27)-dehydrofucreastatin, 5(6),25(27)-disdehydroyuccaloiside C, 5(6)-disdehydroyuccaloiside C, furcreastatin and yuccaloiside C.

7. The composition of claim 1 wherein the saponins comprise an aglycone selected from kammogenin, manogenin, gentrogenin, hecogenin, tigogenin, sarsapogenin, chlorogenin and gitogenin, and at least one glycosidic selected from glucose, xylose, rhamnose, arabinose and galactose.

8. The composition of claim 1 further comprising at least about 10 wt % inorganic salts and at least about 5 wt % saccharides, based upon the bone dry weight of the composition.

9. The composition of claim 1 further comprising from about 10 to about 25 wt % inorganic salts and from about 5 to about 10 wt % saccharides, based upon the bone dry weight of the composition.

10. The composition of claim 1 comprising at least 90 wt % water soluble solids, based upon the bone dry weight of the composition.

11. The composition of claim 1 comprising less than about 2.0 wt % water insoluble solids, based upon the bone dry weight of the composition.

12. The composition of claim 1 wherein the composition is substantially free from water insoluble solids.

13. The composition of claim 1 wherein the composition is substantially free from hydrophobic solids, proteins, and lipids.

14. The composition of claim 1 wherein the non-woody plant is Hesperaloe funifera, Hesperaloe noctuma, Hesperaloe parviflora, Hesperaloe chiangii, or a mixture thereof.

15. A process for preparing an extract from the biomass of non-woody plants of the genus Hesperaloe comprising the steps of cutting the biomass, milling the biomass, extracting the biomass with a solvent, and collecting the crude extract.

16. The process of claim 15 wherein the solvent is an aqueous solvent having a pH from about 6.0 to about 8.0.

17. The process of claim 15 further comprising the step of imbibing the biomass with the crude extract.

18. The process of claim 15 further comprising the step of depithing the biomass.

19. The process of claim 15 wherein the non-woody plant is Hesperaloe funifera, Hesperaloe noctuma, Hesperaloe parviflora, Hesperaloe chiangii, or a mixture thereof.

20. The process of claim 15 further comprising the step of concentrating the crude extract and wherein the concentrated crude extract comprises from about 50 to about 95% solids by weight.

21. The process of claim 15 wherein the solvent is water having a pH from about 6.0 to about 7.5 and a temperature from about 40 to about 90° C.

22. The process of claim 15 wherein the step of cutting the biomass comprises passing the biomass through a hammer mill to yield a cut biomass having a particle size ranging from about 1.0 to about 10.0 mm.

23. The process of claim 15 further comprising processing the crude extract to separate the water soluble fraction from the water insoluble fraction.

24. The process of claim 15 wherein the crude extract comprises from about 5 to about 30 wt % saponins, based upon the bone dry weight of the water soluble solids.

25. The process of claim 16 wherein the crude extract comprises 25(27)-dehydrofucreastatin, 5(6),25(27)-disdehydroyuccaloiside C, 5(6)-disdehydroyuccaloiside C, furcreastatin, yuccaloiside C, or a mixture thereof.

26. The process of claim 15 wherein the crude extract comprises at least about 10 wt % inorganic salts and at least about 5 wt % saccharides, based upon the bone dry weight of the water soluble solids.

27. The process of claim 15 wherein the crude extract comprises from about 10 to about 25 wt % inorganic salts and from about 5 to about 10 wt % saccharides, based upon the bone dry weight of the water soluble solids.

28. The process of claim 15 wherein the crude extract comprises less than about 2.0 wt % water insoluble solids.

29. The process of claim 15 further comprising the step of filtering the crude extract to separate the water insoluble solids from the water soluble solids.

30. The process of claim 15 further comprising the steps of mixing the crude extract with a salt and a solvent to form a first solution, adjusting the pH of the first solution to about 7.0 or less, adding at least one phosphate to the first solution to form a precipitate and removing the precipitate.

31. The process of claim 15 further comprising the steps of mixing the crude extract with a solvent comprising an alcohol and water, defatting the crude extract with a non-polar solvent, and extracting saponins from the defatted crude extract with a polar solvent.
